(19) United States
(12) Patent Application Publication
(10) Pub. No.: US 2015/0005236 A1
(43) Pub. Date: Jan. 1, 2015

(54) USE OF BETA-NERVE GROWTH FACTOR FOR INDUCING OVULATION IN MAMMALS

(71) Applicant: INSTITUT NATIONAL DE LA RECHERCHE AGRONOMIQUE, PARIS CEDEX 07 (FR)

(72) Inventors: Xavier Druart, Montoire Sur Le Loir (FR); Chis Maxwell, New South Wales (AU); Claire Kershaw-Young, Market Drayton (GB)

(73) Assignee: Institut National de la Recherche Agronomique, Paris Cedex (FR)

(21) Appl. No.: 14/376,406

(22) PCT Filed: Feb. 8, 2013
(86) PCT No.: PCT/EP2013/052597

§ 371 Date: Aug. 1, 2014

Related U.S. Application Data
(60) Provisional application No. 61/596,384, filed on Feb. 8, 2012.

(51) Int. Cl. A61K 38/18

(52) U.S. Cl. CPC A61K 38/185
USPC 514/8.4

(57) ABSTRACT

The use of beta-Nerve Growth Factor for inducing ovulation in a mammal. A pharmaceutical or veterinary composition for inducing ovulation in a mammal comprises beta-Nerve Growth Factor in a pharmaceutically acceptable carrier.

### FIELD OF THE INVENTION

The present invention relates to the use of beta-Nerve Growth Factor protein for inducing ovulation in mammals, as well as a pharmaceutical or veterinary composition for inducing ovulation in mammals, comprising beta-Nerve Growth Factor.

### BACKGROUND

In many mammals with induced ovulation, in particular in camelids, the use of assisted reproductive technologies (ARTS) such as artificial insemination (AI) is hindered by a lack of understanding of ovulation induction, the optimal time of AI, and the optimal sperm dose required for successful fertilisation.

In natural mating, camelids are induced to ovulate following copulation. Consequently for AI it is necessary to induce ovulation prior to semen deposition. The current method utilised for ovulation induction in camelids involves transrectal ultrasonography of the ovaries to detect a dominant follicle of suitable size followed by administration of the GnRH analogue Buserelin (Receptal®). In camels, this requires a follicle ranging 1.3-1.8 mm in diameter with 20 μg Buserelin administered intravenously (Skidmore 2011). In alpacas a follicle ranging from 0.6-1.0 mm with 8 μg Buserelin administered intramuscularly (i.m.) is sufficient for ovulation (Vaughan et al. 2003). Semen deposition is generally conducted 24-36 h after ovulation induction (Bravo et al. 2000). Whilst Buserelin is successful in inducing ovulation, ultrasonography is time consuming, requires technical ability and is difficult in llamas and alpacas due to the restricted size of the rectum. Additionally, as farming practices move to a ‘clean, green” approach to farming, there are requirements for a reduction in the use of synthetic hormones for controlled animal breeding programmes.

In camelids, ovulation is induced following mating by a factor present in the seminal plasma of the male, termed ovulation inducing factor. The intrauterine administration of seminal plasma induced ovulation in 87% of camels (Chen et al, 1985), and 41% of alpacas (Ratto et al. 2005) whereas intramuscular (i.m.) administration of seminal plasma induced ovulation in 93% of both alpacas and llamas (Adams el at, 2005; Ratto et at 2005).

Ovulation inducing factor has been characterised as a protein that is different from GnRH, LH, hCG, PMSG and PGF_{2α} (Pan et al. 2001; Paolicchi et al. 1999).

The presence of an ovulation inducing factor in other species, including those that are spontaneous ovulators and those that are induced ovulators, has also been investigated. The seminal plasma of the bull (Ratto et al. 2006), horse, pig (Bogle et al. 2011) and rabbit (Silva et al. 2011) induced ovulation in 26%, 29%, 18% and 100% of llamas respectively. These findings of the prior art suggested that the ovulation inducing factor protein is present in the seminal plasma of many livestock species, but that it is more abundant or most potent in induced ovulators such as the rabbit.

Despite comprehensive research describing the effect of seminal plasma or purified ovulation inducing factor on ovulation, this protein has remained uncharacterised.

There still remains a need for a method for inducing ovulation in camelids and in other mammals, including induced ovulators as well as spontaneous ovulators, that would reduce the need for synthetic hormones in controlled animal breeding programmes, thereby meeting consumer demand for cleaner greener approaches to farming.

It is an object of the present invention to provide such a method.

Additionally, another object of the present invention is to provide that such a method improve ovulation rates and tightly synchronise the timing of ovulation, therefore improving fertility rates following AI in mammals, and in camelids in particular.

### DETAILED DESCRIPTION OF THE INVENTION

The present invention is based on the identification by the present inventors of a major protein component of alpaca seminal plasma which they have found to be able to induce ovulation in female alpacas and in other mammals, particular in both induced ovulators and spontaneous ovulators.

More particularly, alpaca seminal plasma proteins were separated using 10 SOS-PAGE and the most abundant protein of 14 kDa under reducing conditions was identified by the present inventors as beta-Nerve Growth Factor protein (β-NGF) by LC mass spectrometry. The present inventors have identified the ovulation inducing factor protein in alpacas as β-NGF.

The present inventors have in particular discovered that β-NGF is able to induce ovulation in mammals, in particular in alpacas, but also in many other mammals such as ewes, rabbits, cattle, horses, sheep, pigs, goats, camels, that it is equally as successful as the GnRH agonist, Buserelin (Receptal®) and seminal plasma. β-NGF, and that it thus provides an alternative mechanism for the induction of ovulation in mammals, in particular in alpacas, for ARTs, reducing the need for synthetic hormones and possibly improving fertility rates when combining AI with ovulation induction.

Consequently, one aspect of the invention is directed to a method for inducing ovulation in a mammal, comprising administration to said mammal of substantially pure beta-Nerve Growth Factor protein.

According to the invention, said administration may be made by injection, in particular by intramuscular injection.

The mammal may belong either to a species with spontaneous ovulation, such as ewes, or to a species with induced ovulation, such as camelids or rabbits. The mammal may also be a human.

The beta-Nerve Growth Factor protein that is administered to said mammal may be a recombinant protein, in particular a human recombinant protein, or may be purified from seminal plasma, in particular from seminal plasma from a species with induced ovulation, such as camelids or rabbits.

In another aspect, the invention is directed to beta-Nerve Growth Factor protein for its use to induce ovulation in a mammal.

Said beta-Nerve Growth Factor may in particular be administered to said mammal by injection, in particular by intramuscular injection (i.m.). Otherwise it can be administered by subcutaneous injection, intravenous injection, intradermal injection, or by oral administration.

Said mammal may belong to a species with spontaneous ovulation or to a species with induced ovulation.

Beta-Nerve Growth Factor may have been purified from seminal plasma, in particular from a species with induced ovulation, e.g. from a camelid, or it may be a recombinant protein, in particular a human recombinant protein. It may be comprised in a composition further containing a physiologically compatible carrier, in particular a carrier suitable for intramuscular injection.

In some embodiments of the invention, an amount between 0.1 and 10 mg of beta-Nerve Growth Factor is administered to said mammal. Such an amount efficiently induces ovulation both in induced ovulators and in spontaneous ovulators.

Beta-nerve growth factor is a 27 kDa homodimer, which is already known in itself, and which is reduced to two dimers of approximately 14 kDa under reducing conditions such as SDS-PAGE. Human beta-Nerve Growth Factor is identified in the Uniprot database under accession number P01138 (http://www.uniprot.org/uniprot/P01138) and in the NCBI Database under accession number NP_{—}002497.2 (GI:70995319). It is well-conserved among mammals.

Nerve Growth Factor has been implicated in the control of ovarian function (Dissen et al. 1996b) and the Nerve Growth Factor receptor trkA is present in the follicle of the rat (Dissen et al. 1996a). Additionally, nerve growth factor acts through its receptor trkA on human granulosa cells to stimulate the expression of FSH receptors and the secretion of estradiol (Salas et al. 2006).

Nerve Growth Factor has also been purified from bovine seminal plasma as a protein of approximately 15 kDa under reducing conditions (Harper et al. 1982). Concentrations of nerve growth factor in bovine seminal plasma are approximately 0.7 mg/ml of semen and much greater than that in sheep, goat, human and pig seminal plasma (Harper et al. 1982). In contrast, llama seminal plasma contains approximately 125 mg ovulation inducing factor/ejaculate (Tanco et al. 2011). It has been determined by the present inventors (unpublished data) that β-NGF is also present in rabbit seminal plasma.

The i.m. administration of bovine seminal plasma induced ovulation in 26% of llamas (Ratto et al. 2006) and the present inventors have now discovered that this was induced by the presence of β-NGF in bull seminal plasma. The presence of β-NGF in other livestock species has not been reported, however horse and pig seminal plasma induced ovulation in 29 and 18% of llamas respectively (Bogle et al., 2011) and rabbit seminal plasma induced ovulation in 100% of llamas (Silva et al. 2011) when administered i.m. These findings imply that β-NGF is relatively conserved amongst species and it may be concluded that there is some mode of action of seminal plasma β-NGF in spontaneous ovulatory species.

It is likely that the mode of action of seminal plasma β-NGF is at the level of the hypothalamo-pituitary and induces ovulation by stimulating the secretion of LH.

The present inventors have found that, surprisingly, β-NGF acts on GnRH neurons, and more particularly increases the frequency of calcic events, modifies the properties of GnRH neurons network as well as the relations between GnRH neurons and glial cells. These modifications of the properties of the GnRH neurons network by the β-NGF and the synchronization of calcic events are strong evidence that the β-NGF induces GnRH secretion by GnRH neurons. Indeed it was shown that the secretion of GnRH by GnRH neurons was synchronized with calcic events (Constantin et al., 2009)

The source of seminal plasma β-NGF in camelids is unknown. Beta-Nerve Growth. Factor mRNA is expressed predominantly in the vas deferens of the mouse and rat reproductive tract (MacGrogan et al. 1991) whereas in the guinea-pig (Harper et al. 1979; MacGrogan et al. 1991) and bull and rabbit (Harper and Theonen 1980) Nerve Growth Factor is mostly expressed in the prostate. In bactrian camels, intrauterine administration of seminal plasma from a vasectomised male induced ovulation in 100% of females (Chen et al. 1985) suggesting, that ovulation inducing factor is derived from the accessory sex glands and not the testis. It is therefore likely, that seminal plasma β-NGF in camelids is secreted by the prostate.

The invention also relates to a pharmaceutical or veterinary composition for inducing ovulation in mammals, comprising β-Nerve Growth Factor, in particular in the form of a recombinant protein or of a protein purified from seminal plasma, in a pharmaceutically acceptable carrier.

This composition is preferably in a form which can be administered by injection, in particular by intramuscular, subcutaneous, intravenous or intradermal injection, or in a form which can be administered orally.

### BRIEF DESCRIPTION OF THE DRAWINGS

FIG. 1 illustrates a 1D SDS-PAGE of alpaca seminal plasma. 50 μg of protein was separated on a 4-20% acrylamide gel under reducing conditions. The arrow indicates highly abundant protein of about 14 kDa.

FIG. 2 is a graph representing plasma LH concentration, in ng/ml, as a function of time after administration of 1 ml of alpaca's seminal plasma to four ewes in estrous cycle.

FIG. 3 illustrates a Western blot analysis of seminal plasma from boar (lane 1), bull (lane 2), buck (lane 3), ram (lane 4), stallion (lane 5), alpaca (lane 6), camel (lane 7) and human β-NGF as a positive control (lane 8) loaded on a 6-16% SDS-PAGE, blotted, and probed with anti-human β-NGF antibody.

FIG. 4 is a graph representing LH concentration in peripheral blood of female alpacas, in arbitrary units, as a function of time, after i.m. injection of a saline solution (1 ml), Receptal® (4 μg), alpaca seminal plasma (2 ml) or recombinant human β-NGF (1 mg).

FIG. 5 is a graph illustrating the average time between two calcic events in a primary culture of mouse GnRH neurons, before (“control”) and 20 min after application of β-NGF at 75 nM in the culture medium (“β-NGF”).

FIG. 6 represents the individual traces of calcic events of 6 mouse GnRH neurons of a primaryy culture (N1, N2, N5, N9, N16 and N42) (left), before (“control”) and 20 minutes after application of β-NGF at 75 nM in the culture medium; the corresponding raster plots (top right); and the synchronization histogram for treatment with β-NGF at 75 nM (bottom right).

FIG. 7 shows a histogram illustrating the frequency of synchronization events as a function of the % of involved neurons, for mouse GnRH neurons of a primary culture, before (“control”) and 20 minutes after application of 15-NGF at 75 nM in the culture medium.

FIG. 8 shows images obtained by microscopy illustrating the effect of β-NGF on the association of GnRH neurons with glial cells. GnRH neurons were detected with anti-GnRH fluorescent antibodies and glial cells were detected with anti-P75 fluorescent antibodies, in control conditions (“control”) and after 20 min of application of β-NGF at 75 nM in the culture medium (“β-NGF”). “Merge” indicates a merger between the two images of the same row.

### EXAMPLES

These studies were performed using 16 male alpacas. All males were greater than 3 years of age, as at 3 years of age, 100% of alpacas lose their preputial adhesions, testes size reaches a maximum and this is the recommended age for breeding (Tibary and Vaughan 2006).

### Experiment 1

### Identification of Proteins in Alpacas

### Material and Methods

This study was performed using six male alpacas under authorisation from the University of Sydney animal ethics committee. All males were greater than three years of age, had a body condition score greater than or equal to 3, weighed greater than 70 kg and had testes greater than 3 cm in length.

### Seminal Plasma

Semen samples were collected during September 2009 from six males alpacas (3 samples/male) using an artificial vagina fitted inside a mannequin as described previously (Morton et al. 2009). Within 3 min of collection, semen samples were centrifuged for 30 min at 10,000×g, the seminal plasma decanted and the sperm pellet discarded then centrifuged again at 10,000×g for 30 min to ensure all sperm were removed from the ejaculate. Seminal plasma was stored at −80° C. until further analysis.

### One-dimensional SDS-PAGE and Mass Spectrometry

Semen samples were pooled and the protein concentration was determined using the Pierce BCA protein quantification assay (Pierce, Illanois, USA) according to the manufacturer's instructions. Seminal plasma protein (50 μg) was reduced in Laemmli buffer (62.5 mM Tris-HCl pH 6.8 (Sigma-Aldrich, St Louis, Mo., USA), 10% (v/v) glycerol (Sigma), 2% (v/v) sodium dodecyl sulphate (SDS, Sigma), 5% β-mercaptoethanol (Sigma) and 0.2% bromophenol blue at 100° C. for 15 min and separated on a mini protean TGX 4-20% pre-cast SOS-PAGE gel (BioRad, Hercules Calif., USA) for 90 min at 125V. The gel was stained with coomassie blue and the proteins visualised on the GS-800 scanner (Biorad Hercules Calif., USA).

The highly abundant 14 kDa protein was excised from the gel, destained with 50 mM ammonium bicarbonate in 40% acetonitrile, dried, rehydrated with 15 μl modified sequencing grade trypsin (12 ng/μl, Promega, Sydney, Australia) at 4° C. for 1 h, then incubated in 20 μl 50 mM ammonium bicarbonate overnight at 37° C. The sample was analysed using reverse phase LC MS/MS on a Q-STAR Elite mass spectrometer (Applied Biosystems). The peptides were separated on an Agilent 1100 HPLC system using a 30 min gradient of acetonitrile (5-70% in 0.1% formic acid) and eluted peptides were analysed with Analyst OS 1.1 software (Applied Biosystems). The LC MS/MS data were analysed with ProteinPilot 3.0 software (Applied Biosystems) using the uniprot-taxonomy-mammalia database. Only proteins with 95% confidence and at least 2 unique peptides were accepted. Matches to keratin and porcine trypsin and were automatically excluded.

### Results

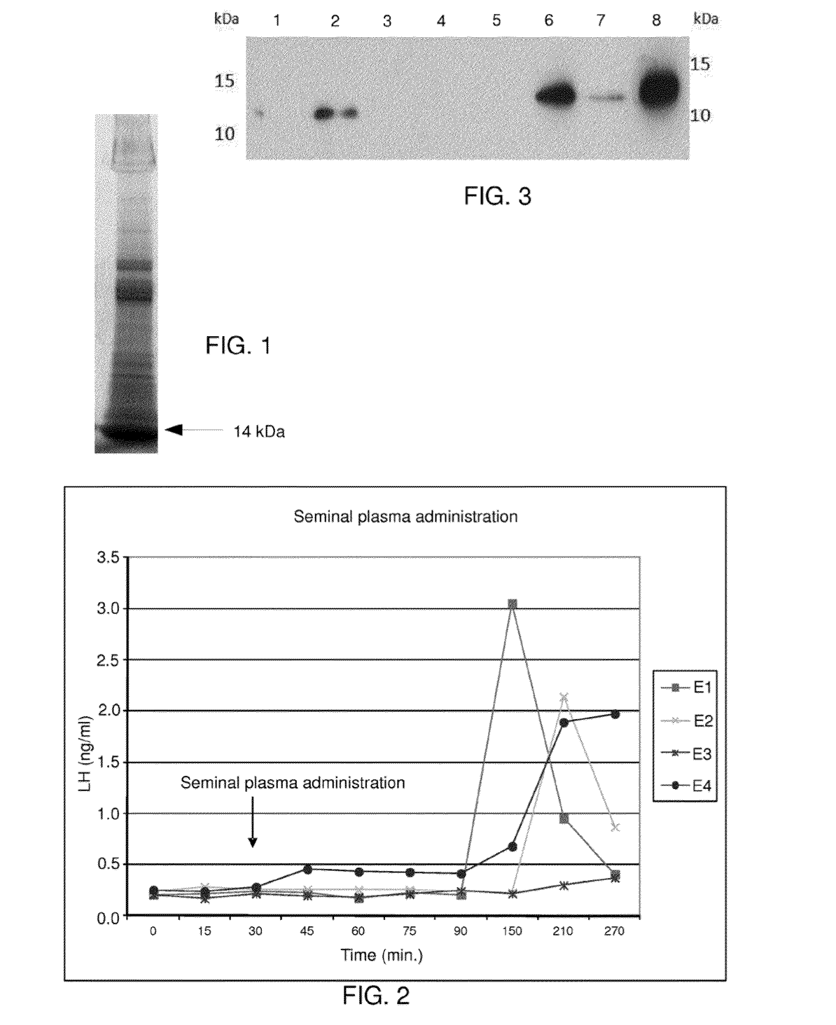

One-dimensional SOS-PAGE of alpaca seminal plasma identified a highly abundant protein of approximately 14 kDa (FIG. 1). Using a protein cut-off of 95% confidence and at least 2 unique peptides, mass spectrometry identified one protein in the 14 kDa protein band: beta-nerve growth factor precursor. The peptide sequences matched those in the data base for bovine (16 peptides), orang-utan (14 peptides), rat (6 peptides) and guinea-pig (4 peptides).

### Experiment 2

### Identification of Proteins in Other Species

### Material and Methods

### Collection and Preparation of Seminal Plasma

Ram (n=3; Merino), bull (n=12; Holstein), goat buck (n=3; Alpine), camel (n=3; Dromedary), horse (n=3; Palouse) and alpaca (n=3; Huacaya) semen were collected using artificial vaginae. Boar semen (n=3; Large White) was collected using the gloved hand technique. Seminal plasma from each species was separated from spermatozoa by centrifugation (10000×g, 10 min, room temperature). The supernatant was centrifuged again (10000×g, 10 min, room temperature) and stored at −80° C.

### Immunodetection of β-NGF in Seminal Plasma

Beta-Nerve Growth Factor (β-NGF) was detected by western blotting by loading on a 6-16% SOS-PAGE, blotting, and probing with anti-human β-NGF antibody.

The presence of β-NGF was searched in the seminal plasma from the several mammalian species including cattle, horse, sheep, pig, goat, camel and alpaca. The affinity of the antibody directed against human β-NGF toward seminal β-NGF was very different between species. Therefore the amount of seminal plasma proteins deposited on each lane of the gel had to be adjusted to allow detection or avoid saturation. The amounts were 50 μg, 10 μg, 50 μg, 50 μg, 50 μg, 1.6 ng and 1.6 ng for respectively, boar, bull, ram, buck, stallion, alpaca and camel seminal plasma. Human recombinant beta-Nerve Growth Factor obtained commercially (7.6 ng) was used as a control.

Semidry transfer of proteins was performed over 1.5 h at 0.8 mA/cm². The western blots were blocked with TBS-Tween® 20 (0.5% w/v), supplemented with lyophilized low-fat milk (5%, w/v). Membranes were incubated with rabbit polyclonal antibodies directed against human β-NGF (1/5000 v/v, sc548, Santa Cruz) under mild agitation overnight at 4° C. The second antibody was a goat anti-rabbit conjugated with peroxidase (dilution 1:5000). The peroxidase was revealed with chemoluminescent substrates and the images recorded on film or digitized with a cooled CCD camera. No reaction was observed with the secondary antibodies alone.

### Results

Using the antibody directed against human NGF, the presence of β-NGF was further confirmed by Western blot in alpaca, camel and bull seminal plasma (FIG. 3). No cross reactivity could be detected with ram and stallion NGF.

β-NGF was detected in the seminal plasma of bull, ram, stallion, alpaca and camel by 2D LC MS/MS. A strong immunoreaction at approximately 13 kDa was observed in alpaca, camel and bull seminal plasma.

### Experiment 3

### Induction of Ovulation in Alpacas

### Material and Methods

### Animals

This study was performed using 20 female alpacas under authorisation from the University of Sydney animal ethics committee. Females were housed in pens of 5 with hay and water provided ad libitum. All females had previously had at least 1 cria, were greater than 4 years of age (mean 111±7.9 months) and were reproductively active.

### Treatments

The emergence of dominant follicles was synchronised with the 0.8 ml (200 μg) prostaglandin F_{2α} (Estrumate®, Intervet) i.m. followed by 1 ml (4 μg) of Buserelin (Receptal®, Intervet) i.m. 24 h later, then luteolysis was induced 11 days after induced ovulation with 0.8 ml Estrumate®.

To determine the presence of a dominant follicle, females were restrained in sternal recumbency and examined by transrectal ultrasonography using an Aloka SSD-500 scanner with 7.5 MHz linear array transducer (Aloka Co., Japan). The ovaries of each follicle were scanned. Females with a newly emerged dominant follicle of 6-10 mm in diameter were randomly assigned to receive 1 of 4 treatments: (1) 1 ml 0.9% saline in, (n 5), (2) 1 ml (4 μg) Receptal® (Intervet) i.m (n=5), (3) 2 ml alpaca's seminal plasma i.m. collected as described above for Experiment ˜1 (n=5), (4) 1 mg recombinant active human β-NGF protein (Sino Biological, Japan) Lm. (n=5).

Animals were examined by transrectal ultrasonography 28-30 h following treatments to detect ovulation. Ovulation was defined as the absence of the dominant follicle that was observed during the previous scan at the time of treatment. Ultrasonography was also repeated 8 days following treatment, to confirm ovulation and determine the size of the corpus luteum.

### Blood Samples and Hormone Analysis

Female alpacas were cannulated and jugular venous blood samples (5 ml) were collected for detection of luteinising hormone immediately before treatment, every 30 min for 4 h, then every 1 h for 4 h and finally at 12 h post-treatment. Jugular venous blood samples (5 ml) were also collected on day 8 at the time of ultrasonography for detection of plasma progesterone. Samples were placed in heparinised tubes, centrifuged at 2000×g for 10 min, the plasma decanted and stored at −20° C. until analysis.

Plasma LH concentration were determined using a RIA. Plasma progesterone concentrations were determined using a commercial double antibody RIA kit (Coat-a-Count).

### Statistical Analysis

Data were analysed using ANOVA in GENSTAT (Version 11, VSN

International, Hemel Hempstead, UK) with post-hoc comparisons using the least significant difference (LSD) test where appropriate. Far all analyses P<0.05 was considered significant. To determine differences in plasma progesterone concentrations between treatments, animals in the Receptal®, seminal plasma and β-NGF treatment groups that did not ovulate were excluded from the analysis.

### Results

In this experiment, female alpacas (n 5/group) were administered i.m. with (1) 1 ml 0.9% saline, (2) 1 ml of the GnRH agonist Receptal® (3) 2 ml alpaca seminal plasma, or (4) 1 mg human β-NGF. Ovulation was detected by transrectal ultrasonography 8 days following treatment and confirmed by plasma progesterone concentrations.

The results are indicated in Table 1:

TABLE 1
Follicle diameter (mm) pre-treatment, and corpus luteum (CL)
diameter (mm) and plasma progesterone concentrations (ng/ml) on day 8
following treatment in females alpacas administered 1 ml 0.9% saline, 1 ml
(4 μg) Buserelin (Receptal ®), 2 ml alpaca seminal plasma and 1 mg human
β-NGF i.m. Plasma progesterone concentrations do not include data from
animals that did not ovulate, except for saline-treated animals.

| | | | Progesterone |
|---|---|---|---|
| Treatment Group | Follicle Diameter | CL diameter | conc. |
| Saline | 8.0 ± 0.63^{a} | None present | 0.12 ± 0.015^{a} |
| Receptal | 8.8 ± 0.73^{a} | 9.3 ± 1.49^{a} | 4.01 ± 0.897^{b} |
| Seminal plasma | 8.2 ± 0.80^{a} | 9.3 ± 1.25^{a} | 2.44 ± 0.717^{b} |
| β-NGF | 8.0 ± 1.22^{a} | 10.3 ± 1.03^{a} | 3.28 ± 0.681^{b} |

^{a, b}within a column, values without a common superscript differ significantly (P < 0.05).
Values are mean ± sem.

As shown in Table 1, when scanned for the presence or absence of the dominant follicle at 28-30 h following treatment with Receptal®, seminal plasma and β-NGF, the dominant follicle was absent in 80, 60 and 80% of female alpacas respectively. However, in saline-treated alpacas, the dominant follicle was present in 5/5 animals suggesting ovulation had not occurred. On day 8 following treatment corpus lutes were detected in 0, 80, 80 and 80% of alpacas treated with saline, Receptal®, seminal plasma and β-NGF respectively.

The size of the dominant follicle on the day of treatment did not differ between treatment groups (P=0904, Table 1). The size of the corpus luteum on day 8 following treatment did not differ (P=0.818) between animals treatment groups (Table 1). Plasma progesterone concentrations differed significantly between treatment groups (P=0.003) and were higher in alpacas treated with Receptal®, seminal plasma and β-NGF, than those treated with saline (table 1).

These results demonstrate that i.m. administration of β-NGF in alpacas induce ovulation with equal success to seminal plasma or Receptal®. Additionally, the treatment did not affect corpus luteum size 8 days after treatment, suggesting that all treatments initiated a similar ovulatory effect.

### LH Concentration in Peripheral Blood

The evolution of the concentration of LH in peripheral blood was assessed for 12 hours after the injections. The negative control (saline) did not induce any significant increase of LH whereas all three experimental treatments (Receptal®, β-NGF, seminal plasma) induced a significant increase of LH concentration with a very similar response over the time (FIG. 4). Interestingly, the delay of response was the same for the human recombinant β-NGF, the camelid β-NGF from the seminal plasma and the GnRH, with a peak of LH 3 hours after injection.

It is thus demonstrated that β-NGF initiates an LH surge within 8 h of treatment.

### Experiment 4

### Induction of Ovulation in Ewes

1 ml of alpaca's seminal plasma obtained as described in Experiment 1 was administered by intramuscular injection to four ewes in estrous cycle, called E1 to E4. Before seminal plasma injection, the concentration of plasma LH was zero for all these ewes.

Changes in plasma LH concentration over time was then determined according to the protocol described in Experiment 2. The results are shown on FIG. 2. This graph shows LH secretion by the pituitary within 4 hours following seminal plasma injection, demonstrating that alpaca's seminal plasma, containing a high level of β-NGF, induces ovulation in ewes, a species with spontaneous ovulation,

### Experiment 5

### Induction of Ovulation in Female Rabbits

### Material and Methods

A herd of 83 female rabbits of proven fertility was used in this experiment. These female rabbits were distributed into 4 comparable groups (number of litters already had, weight, number of weaned young rabbits in the previous litter):
group GnRH (as a positive control): the female rabbits receive an i.m. injection of 0.2 ml of Fertagyl® containing 20 μg of GnRH (Gonadotrophine Releasing Hormone)
group saline (as a negative control): the female rabbits receive an i.m. injection of 0.2 ml of physiological saline (sterile solution of sodium chloride at 0.9% in water)
group PS (experimental group): the female rabbits receive an i.m. injection of 0.2 ml of pure alpaca seminal plasma
group β-NGF (experimental group): the female rabbits receive an i.m. injection of 50 μg of recombinant human β-NGF in 0.2 ml of physiological saline.

The female rabbits were synchronized by an injection of eGG 48 hours before insemination. Then they were inseminated with a mixture of semen from several males. The same mixture was used for all females.

The female rabbits were killed 48 hours after insemination. The ovaries were collected to proceed to counting the corpora lutea, pre-ovulatory follicles of diameter higher than 1 mm and atretic follicles, and to identify potential pseudopregnant females. The oviducts were infused in order to count oocytes and embryos and to evaluate the developmental stage of the latter (number of blastomeres of the embryo).

### Statistical Data Analysis

The results were analysed with the software SAS using a fixed-effects variance analysis, taking into account the fixed effect of the treatment (4 levels: GnRH, β-NGF, PS and saline).

### Results

The results of the variance analysis, with estimated mean values, for ovulation frequency and intensity, and number of collected morulae, are indicated in Table 2.

TABLE 2
Influence of the treatment on the ovulation frequency and intensity
and on the number of collected morulae for the 4 groups of female
rabbits - results of variance analysis, estimated mean values.

| | Mean | R² | Group | GnRH | β-NGF | PS | Saline |
|---|---|---|---|---|---|---|---|
| Number | 83 | | | 22 | 20 | 20 | 21 |
| Parity | 5.33 | 0.032 | NS | 5.73 | 5.70 | 5.05 | 4.81 |
| Weaned | 6.66 | 0.009 | NS | 6.45 | 7.05 | 7.00 | 6.19 |
| number | | | | | | | |
| Weight of | 4900 | 0.022 | NS | 4933 | 4824 | 4751 | 4986 |
| rabbit (g) | | | | | | | |
| Corpora | 5.53 | 0.518 | P < 0.001 | 13.82^{a} | 3.30^{b} | 3.00^{b} | 1.38^{b} |
| lutea⁽¹⁾ | | | | | | | |
| Haemorrhagic | 0.64 | 0.080 | P = 0.042 | 1.36^{a} | 0.70^{ab} | 0.25^{b} | 0.19^{b} |
| follicles | | | | | | | |
| Collected | 4.88 | 0.483 | P = 0.084 | 12.09^{a} | 3.05^{b} | 2.70^{b} | 1.14^{b} |
| embryos | | | | | | | |
| Ovulation (%) | 39.76 | 0.559 | P < 0.001 | 100^{a} | 25.0^{b} | 20.0^{b} | 9.5^{b} |

NS = not significant
⁽¹⁾number of corpora lutea by injected female rabbit
^{a,b}Within a row, values without a common superscript differ significantly

The 4 groups did not show significant differences in the distribution parameters (parity, weight and number of young individuals in the previous litter), demonstrating, that the females had been appropriately distributed in the groups.

The results for groups GnRH et saline were as expected, with 100% of ovulation for GnRH (positive control) and 9.5% of ovulation for the saline solution (negative control).

Treatment with alpaca's seminal plasma and β-NGF induced an increase of the ovulation rate with respect to the negative control (saline) with 20% and 25% of ovulation, respectively.

β-NGF induced an increase in the number of corpora lutea (3.3 vs 1.38) and haemorrhagic follicles (0.7 vs 0.19) with respect to the serum.

Ovulations induced by the β-NGF protein lead to the production of oocytes of normal fertility, since the number of produced embryos is also higher than for the saline control (3.05 vs 1.14).

These results demonstrate the efficiency of β-NGF to induce ovulation in female rabbits.

### Experiment 6

### Effect of β-NGF on GnRH Neurons in Mice

### Material and Methods

### In Vitro Model—Primary Culture of GnRH Neurons

A model of primary culture of GnRH neurons has been developed from explants of olfactory placodes of mouse embryos (Constantin et al., 2009). This model has three stages of in vitro development: from 1 to 3 days, intra-explant migration, corresponding to an in vivo intranasal migration; from 3 to T days, out-of explant migration, corresponding to an in vivo intracerebral development; from 7 to 15 days, occurrence of the pulsatile secretion, corresponding to neuronal and network maturation.

This experiment was carried out between 7 and 15 days of in vitro development corresponding to a stage of mature GnRH neurons having a pulsatile GnRH secretion.

Primary Culture from Nasal Explants

Embryos were obtained from timed pregnant animals. Nasal pits of embryonic d 11.5 staged Swiss mice were isolated under aseptic conditions and refrigerated for 1 h in Gey's balanced salt solution (Eurobio, Les Ulis, France) enriched with glucose (Sigma-Aldrich Corp., St. Louis, Mo.). Nasal explants were adhered onto coverslips by a chicken plasma (local source)/thrombin (Sigma-Aldrich) clot. The explants were maintained in a defined serum-free media (SFM). On culture d 3, fresh media containing uridine (5 mg/ml; Sigma-Aldrich) and 5′-fluoro-2-deoxyuridine (2 mg/ml; Sigma-Aldrich) were given to inhibit proliferation of dividing olfactory neurons and non-neuronal explant tissue. On culture d 6 and every 2 d afterward, the media were changed to fresh SFM. Explants were used for experiments from 3-21 div, encompassing developmental stages known to exhibit synchronization of calcium oscillations in GnRH-1 neurons.

### Analysis of the Calcic Neuronal Signaling

Variations of intra-cellular free calcium ([Ca²⁺]) concentration reflect the endogenous and synaptic activity of neurons.

These variations were measured by “time lapse” imaging in the primary culture of GnRH neurons. A quantitative analysis was carried out for single neurons but also overall for a group of neurons constituting part of the network.

Calcium imaging recordings were performed between 6 and 10 div. The Calcium Green-1 AM (Molecular Probes) was diluted to a 2.7-mm concentration in 80% dimethylsulfoxide and 20% pluronic F-127 solution (Molecular Probes). This solution was diluted 1:200 with SFM (serum-free media) to a final Calcium Green-1 concentration of 13.5 μm.

Nasal explants, maintained at 37° C. in a 5% CO₂ humidified incubator, were incubated with this loading solution for 20-30 min, then washed twice with fresh SFM (10 min each). Explants were mounted into a perfusion chamber and were continuously superfused with medium, at a rate of approximately 60 μl/min, using a peristaltic pump. The perfusion chamber was maintained at 36 C using a temperature controller (Warner Instruments, Hamden, Conn.).

Calcium Green-1 was visualized using an inverted microscope (DM-IRB; Leica. Microsystems GmbH, Wetzlar, Germany), through a ×20 fluorescence objective, and acquired using a cooled intensified charge-coupled device camera (CoolSNAP fx; Rapper Instruments, Photometrics, Tucson, Ariz.). Experiments were piloted by Metafluor (Molecular Devices, Downingtown, Pa.), controlling the shutter and the acquisition (every 20 sec for 3 h), Excitation wavelengths were provided through a medium-width excitation bandpass filter at 465-495 nm, and emission was monitored through a 40-nm bandpass centered on 535 nm. Fluctuations in [Ca2+]i were analyzed a posteriori with Metafluor software. Each cell, individually identified, was circled. Calcium Green-1 fluorescence intensity was plotted and analyzed with Excel (Microsoft Corp., Redmond, Wash.). All recordings were terminated by a 40-mm KCl stimulation to ensure the viability of the recorded cells, increasing basal secretion by 2.5-fold.

Acquired every 20 sec, a calcium peak was defined when a value was greater than 1.5×sd on the average of the five previous and five subsequent points. Synchronicity was defined as the percentage of cells exhibiting a peak simultaneously.

### Phenotypic Characterization of Cells

An immunohistochemical approach was used to characterize the cells present in the culture, using anti-GnRH antibody for the GnRH neurons, anti-S100b antibody for the glial cells and anti-P75 antibody (P75 is a NGF-specific receptor). The analysis was carried out by confocale microscopy.

### Immunocytochemistry

Nasal explants were fixed in 4% formaldehyde [45-60 min, rinsed in PBS (3×10 min)], blocked (1 h, 10% normal goat serum, 0.3% Triton X-100, and 0.1% sodium azide), rinsed in PBS (3×8 min), and incubated overnight in anti-GnRH-1 (1:3000, SW-1). Antibodies were diluted in PBS containing 10% normal goat serum, 0.3% Triton X-100, and 0.1% sodium azide. The next day, the explants were rinsed in PBS (3×8 min), incubated in secondary antibody ant rabbit Alexa 546 (1:500, 1 h; Molecular Probes, Inc., Eugene Oreg.) and rinsed in PBS (4×8 min). Explants incubated with fluorescent dies were mounted with Vector Shield (Vector Laboratories).

### Results

### 1/β-NGF Increases Individual Activity of GnRH Neurons and the Overall Activity of the GnRH Neurons Network

1.1/β-NGF Increases the Frequency of Calcic Events

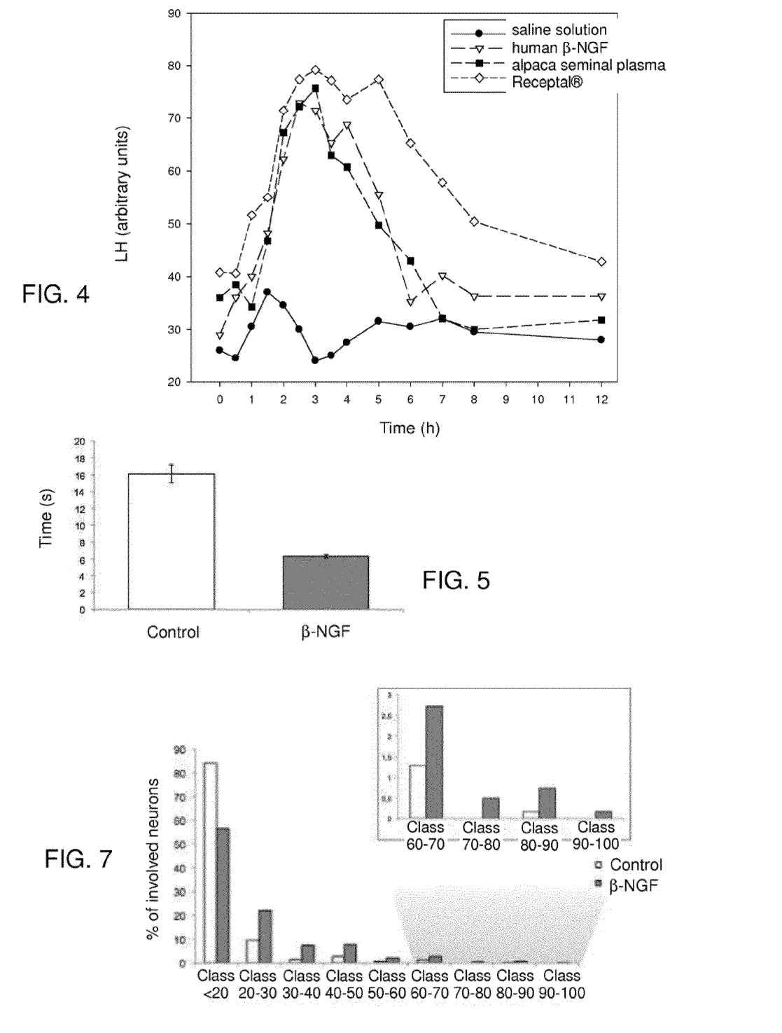

FIG. 5 shows the average time between two calcic events before and 20 min after application of β-NGF at a concentration of 75 nM in the culture medium.

As it can be seen, the average time between two events is about 15 s. After 20 min of application of β-NGF at a concentration of 75 nM in the culture medium, this time is divided by 3, which is highly significant.

Application of β-NGF at 75 nM thus leads to an increase of the frequency of calcic events, and therefore to an increase of neuronal activity.

1.2/β-NGF modifies the properties of GnRH neurons network

The synchronization of calcic events, i.e. the percent of recorded neurons having a calcic event at the same time, was measured. The calcic events were detected by a maximum detection method.

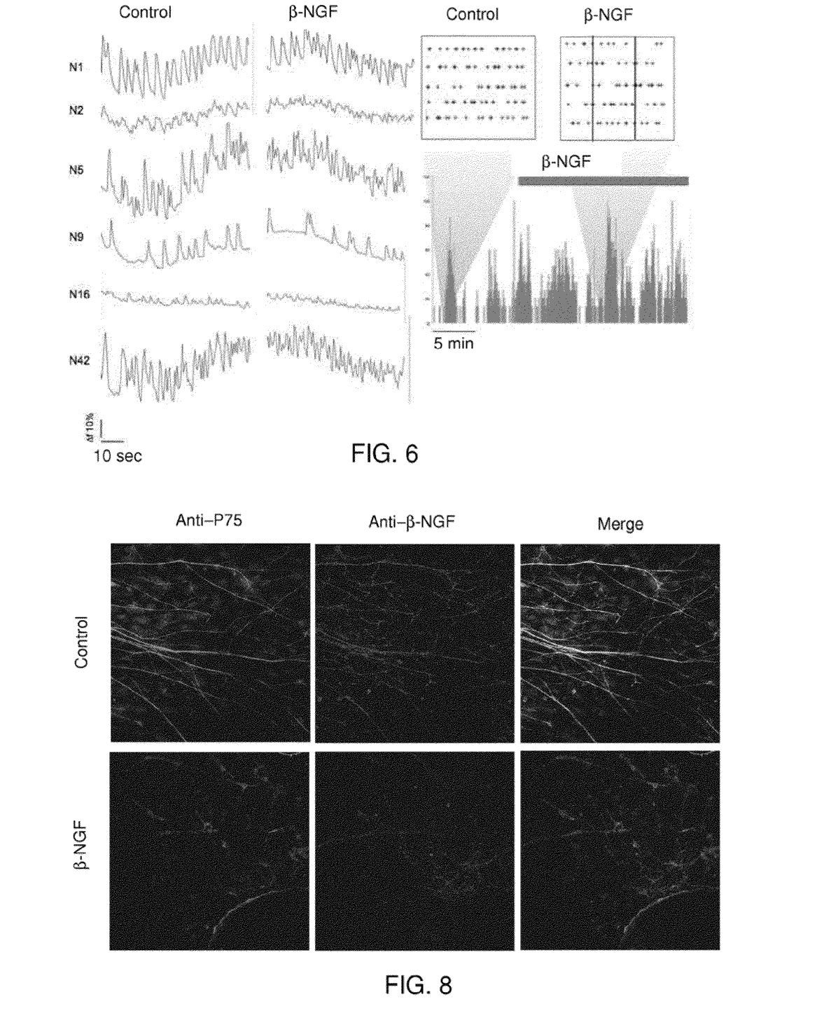

The results are shown on FIG. 6. On this figure, the individual traces of 6 GnRH neurons (N1, N2, N5, N9, N16 and N42) are represented on the left, before (“control”) and 20 minutes after application of β-NGF at 75 nM in the culture medium. The corresponding raster plots are represented on the right at the top, and the synchronization histogram for treatment with β-NGF at 75 nM on the right at the bottom. These data show that the synchronization of calcic events between GnRH neurons was increased by the β-NGF. The β-NGF acts a stimulus enhancing a synchronized activity of these neurons.

The analysis of the distribution of synchronization events, illustrated in FIG. 7, shows that the synchronization increase concerns essentially the events involving more than 70% of the neurons, i.e. the synchronization events representing less than 5% of the total synchronization events.

The application of β-NGF at 75 nM in the culture medium leads to an increase of the percent of synchronization events involving more than 70% of the recorded GnRH neurons, demonstrating that β-NGF at 75 nM modifies the activity of the GnRH neurons network.

### 2/Architectural Features of the Network

2.1/β-NGF receptor P75 is expressed by glial cells and GnRH neurons P75, a β-NGF receptor, is mainly expressed by the glial cells associated with GnRH neurons, but also by the neurons themselves.

Immunohistochemical analysis of the cells with anti-P75 antibody (FIG. 8) show that both glial cells and GnRH neurons have β-NGF receptors and show a close association.

2.2/β-NGF modifies the relations GnRH neurons—glial cells

The relations between GnRH neurons and glial cells have been analyzed by immunohistochemistry after 20 min of recording in calcic imaging, for the control and after 20 min of application of β-NGF at 75 nM in the culture medium, and after K⁺ stimulation. The glial cells retract after K⁺ stimulation in the presence of β-NGF, but not in control conditions. This suggests that β-NGF at 75 nM acts on glial plasticity at proximity of GnRH neurons.

As can be observed on FIG. 8, the glial extensions shorten after K⁺ stimulation in the presence of β-NGF at 75 nM in the culture medium. This reduction of the length of the extensions is indicative of a plasticity induced by the β-NGF. This plasticity is an indication of the change of activity of the GnRH neurons induced by the β-NGF

### REFERENCES

Adams, G. P., Ratto, M. H., Huanca, W., and Singh, J. (2005). Ovulation-Inducing Factor in the Seminal Plasma of Alpacas and Llamas. Biol. Reprod. 73, 452-457.
Bogle, O. A., Ratto, M. H., and Adams, G. P. (2011). Evidence for the conservation of biological activity of ovulation-inducing factor in seminal plasma. Reproduction 142, 277-283.
Bravo, P. W., Skidmore, J. A., and Zhao, X. X. (2000). Reproductive aspects and storage of semen in Camelidae. Anim. Reprod. Sci, 62, 173-193,
Chen, B. X., Yuen, Z. X., and Pan, G. W. (1985). Semen-induced ovulation in the Bactrian camel (Camelus bactrianus) J. Reproof. Fertil, 73, 335-339.
Constantin, S., Caraty A., Wray S., and Duittoz. A. Development of Gonadotropin Releasing Hormone-1 Secretion in Mouse Nasal Explants. Endocrinology. 2009 July; 150(7): 3221-3227.
Dissen, G. A., Hill, D. F., Costa, M. E., Dees, W. L., Lara, H. E. and Ojeda, S. R. (1996a). A role for trkA nerve growth factor receptors in mammalian ovulation. Endocrinology 131, 198-209.
Dissen, G. A., Mayerhofer, A., and Ojeda, S. R. (1996b). Participation of nerve growth factor in the regulation of ovarian function. Zygote 4, 309-312.
Harper, G. P., Bate, Y. A., Burnstock, G., Carstairs, J. R., Dennison, M. E., Suda, K., and Vernon, C. A, (1979). Guinea pig prostate is a rich source of nerve growth factor. Nature 279, 160-162.
Harper, G. P., Glanville, R. W., and Thoenen, H. (1982). The Purification of Nerve Growth Factor from Bovine Seminal Plasma. Biochemical characterization and partial amino acid sequence. The Journal of Biological Chemistry 257, 8541-8542.
Harper, G. P., and Theonen, H. (1980). The distribution of nerve growth factor in the male sex organs of mammals. J. Neurochem. 34, 893-903.
MacGrogan, D., Despres, G., Romand, R., and Dicou, E. (1991). Expression of the B-Nerve Growth Factor Gene in Male Sex Organs of the Mouse, Rat, and Guinea Pig. J. Neurosci. Res. 28, 567-573.
Morton, K. M., Gibb, Z., Bertoldo, M., and Maxwell, V, M. C. (2009). Effect of diluent, dilution rate and storage temperature on longevity and functional integrity of liquid stored alpaca (Vicugna Pacos) semen. Journal of Camelid Science 2, 15-25.
Pan, G., Chen, Z., Liu, X., Li, D., Xie, Q., Ling, F., and Fang, L. (2001). Isolation and Purification of the Ovulation-Inducing Factor from Seminal Plasma in the Bactrian Camel (camelus bactrianus). Theriogenology 55, 1863-1879.
Paolicchi, F., Urquieta, B., Del Valle, L., and Bustos-Obregon, E. (1999). Biological activity of the seminal plasma of alpacas: stimulus for the production of LH by pituitary cells. Anim. Roprod. Sci. 54, 203-210.
Ratio, M. H., Huanca, W., Singh, J., and Adams, G. P. (2005). Local versus systemic effect of ovulation-inducing factor in the seminal plasma of alpacas. Reproductive Biology and Endocrinology 3.
Ratto, M. H., Huanca, W., Singh, J., and Adams, G. P. (2006). Comparison of the effect of ovulation-inducing factor (OIF) in the seminal plasma of llamas, alpacas, and bulls. Theriogenology 66, 1102-1106.
Salas, C., Julio-Pieper, M., Valladares, Pommer, R., Vega, M., Mastronardi,

C., Kerr, B., Ojeda, S. R., Lara, H. E., and Romero, C. (2006). Nerve Growth Factor-Dependent Activation of trkA Receptors in the Human Ovary Results in Synthesis of Follicle-Stimulating Hormone Receptors and Estrogen Secretion. The Journal of Clinical Endocrinology and Metabolism 91, 239602403,
Silva, M., Nino, A., Guerra, M., Letelier, C., Valderrama, X. P., Adams, G. P., and Ratto, M. H. (2011). Is an ovulation-inducing factor (OIF) present in the seminal plasma of rabbits? Anim. Reprod. Sci. 127, 213-221.
Skidmore, J. A. (2011). Reproductive Physiology in Female Old World Camelids. Anim. Roprod. Sci, 124, 148-154.
Tanco, V. M., Ratto, M. H., Lazzarotta, M., and Adams: G. P. (2011). Dose-Response of Female Llamas to Ovulation-Inducing Factor from Seminal Plasma. Biol. Reprod. 85, 452-456.
Tibary, J., and Vaughan, J. (2006). Reproductive infertility and physiology in male South American camelids: A review and clinical observations. Small Ruminant Research 61, 283-298.
Vaughan, J., Galloway, D., and Hopkins, a (2003) ‘Artificial insemination in Alpacas (Lama Pacos).’ (Rural Industries Research and Development Corporation: Kingston, ACT, Australia)

1-8. (canceled)

9. A pharmaceutical or veterinary composition for inducing ovulation in a mammal, comprising beta-Nerve Growth Factor in a pharmaceutically acceptable carrier.

10. A pharmaceutical or veterinary composition as claimed in claim 9, in a form which can be administered by injection.

11. A method for inducing ovulation in a mammal, comprising administration of substantially pure beta-Nerve Growth Factor to said mammal.

12. A method as claimed in claim 11, wherein said administration is made by injection.

13. A method as claimed in claim 12, wherein said administration is made by intramuscular injection.

14. A method as claimed in claim 11, wherein said mammal belongs to a species with spontaneous ovulation.

15. A method as claimed in claim 11, wherein said mammal belongs to a species with induced ovulation.

16. A method as claimed in claim 11, wherein said beta-Nerve Growth Factor is purified from seminal plasma.

17. A method as claimed in claim 16, wherein said beta-Nerve Growth Factor is purified from seminal plasma from a species with induced ovulation.

18. A method as claimed in claim 11, wherein said beta-Nerve Growth Factor is a recombinant protein.

19. A method as claimed in claim 11, wherein said beta-Nerve Growth Factor is administered to said mammal in an amount between 0.1 to 10 mg.

20. A pharmaceutical or veterinary composition as claimed in claim 9, in a form which can be administered by intramuscular injection.

21. A pharmaceutical or veterinary composition as claimed in claim 9, wherein said beta-Nerve Growth Factor is purified from seminal plasma.

22. A pharmaceutical or veterinary composition as claimed in claim 9, wherein said beta-Nerve Growth Factor is purified from seminal plasma from a species with induced ovulation.

23. A pharmaceutical or veterinary composition as claimed in claim 9, wherein said beta-Nerve Growth Factor is a recombinant protein.
